(19) United States
(12) Patent Application Publication
(10) Pub. No.: US 2011/0314726 A1
(43) Pub. Date: Dec. 29, 2011

(54) PRODUCTION OF ETHANOL FROM LIGNOCELLULOSIC BIOMASS USING GREEN LIQUOR PRETREATMENT

(76) Inventors: Hasan Jameel, Cary, NC (US); Richard Phillips, Raleigh, NC (US); Hou-Min Chang, Raleigh, NC (US); Yongcan Jin, Jiangsu (CN)

(21) Appl. No.: 13/130,516

(22) PCT Filed: Nov. 23, 2009
(86) PCT No.: PCT/US09/65564

§ 371 Date: Sep. 16, 2011

Related U.S. Application Data
(60) Provisional application No. 61/116,934, filed on Nov. 21, 2008.

(51) Int. Cl. C10L 1/182; C12P 7/10; C12P 7/02

(52) U.S. Cl. 44/451; 435/155; 435/165

(57) ABSTRACT

A method of producing an alcohol from lignocellulosic biomass is provided wherein the lignocellulosic biomass is pretreated with an alkaline mixture of sodium carbonate and sodium sulfate, i.e., green liquor, prior to enzymatic hydrolysis and fermentation. Pretreatment with the green liquor increases the efficiency of the enzymatic hydrolysis. Both the alcohol produced from the fermentation and the lignin that dissolves into the green liquor during pretreatment can also be used as fuels.

### RELATED APPLICATIONS

This application claims the benefit of U.S. Provisional Patent Application No. 61/116,934, filed Nov. 21, 2008, the disclosure of which is incorporated herein by reference in its entirety.

### TECHNICAL FIELD

Methods of pretreating lignocellulosic biomass with green liquor and of producing alcohol from lignocellulosic biomass using a green liquor pretreatment step are provided.

ABBREVIATIONS

| | |
|---|---|
| ° C.= | degrees Celsius |
| %= | percentage |
| % K= | fiber consistency |
| FPU= | filter paper units |
| g= | gram |
| GL= | green liquor |
| HS⁻= | hydrosulfide |
| L= | liter |
| min= | minutes |
| Na₂CO₃= | sodium carbonate |
| NaOH= | sodium hydroxide |
| Na₂S= | sodium sulfide |
| Na₂SO₄= | sodium sulfate |
| OD= | oven dried |
| S²⁻= | sulfide |
| SSF= | simultaneous saccharification |
| | and fermentation |
| TTA= | total titratable alkali |

### BACKGROUND

Plant-derived lignocellulosic biomass represents a large, renewable source of potential starting materials for the production of a variety of chemicals, plastics, fuels and feeds. For example, lignocellulosic biomass feedstocks comprise cellulose, which can be hydrolyzed to provide fermentable sugar for use in the production of ethanol.

Currently, large scale production of bioethanol (i.e., ethanol produced from biomass) relates to two sources: sugarcane from Brazil, which is squeezed to provide a sucrose rich juice which can then be fermented into alcohol, and corn grain from the midwestern region of the United States, which is processed with amylase to convert starch into fermentable sugars that can be fermented into alcohol. Even though many types of woody biomass (e.g., loblolly pine thinnings, natural hardwoods, and the harvesting residues associated with these commercially common wood resources) are fully competitive with sugarcane and corn grain in terms of growth rate and overall feedstock cost, little bioethanol is currently produced from such sources. Some of the reasons for this situation are: (1) the physical density of wood; and (2) the chemical complexity of wood. The first factor can make penetration of the biomass structure of wood with chemicals difficult, while the second factor can lead to difficulty in breaking down the long chain polymeric structure of cellulose into monomeric sugars that can be fermented.

During both World Wars, when gasoline fuel was in short supply, many countries, including the United States, Germany, Russia and Sweden, developed processes involving strong acids to breakdown the polymers in wood to produce fermentable sugars. When the shortages ended, wood-to-ethanol production stopped due to the poor economics of the wood-to-ethanol process compared to the production costs of more common automotive fuels, such as gasoline and diesel.

In view of more recent energy source concerns, alternative biomass-to-ethanol processes have been developed, including the so-called “dilute acid process,” in which lignocellulosic biomass is mixed with sulfuric acid and fed into a pressurized vessel that holds the acid-biomass mixture for a short period of time. It is critical that the right concentration of acid be used for the right period of time in order to break chemical bonds and release hemicellulosic material (e.g., mannans and xylans) in the wood matrix, but to minimize side reactions that convert portions of the dissolved material into furfural. Furfural formation reduces the potential yield of ethanol and inhibits the fermentation process. Because of the detrimental effect of furfural, process equipment must be employed to physically remove the compound before further processing. The solid residue that remains after the first acid treatment is then mixed with a stronger sulfuric acid solution and fed into a second pressurized vessel that holds the resulting slurry for a longer period of time in order to break down remaining cellulosic materials into monomeric sugars. In addition to monomeric sugar, the product of the second acid step comprises undissolved lignin residue and degraded or partially degraded cellulosic material (e.g., oligosaccharides). The overall process requires two distinct pressure vessels and feed systems and must be constructed from relatively expensive metallurgical alloys (e.g., zirconium) in order to withstand the processing conditions. The products of the two acid treatment steps are neutralized with lime. The resulting neutralization side product, calcium sulfate (also referred to as gypsum), can then need to be disposed of.

Typically, the two acid treatment steps are not sufficiently severe as to cause complete destruction of all of the polymeric cellulose bonds, thus the acid treatment steps are followed by treatment with enzymes (e.g., cellulase) to complete the breakdown (e.g., the hydrolysis) of all of the cellulose into sugars that can be fermented. Fermentation converts the sugars to ethanol. Since the ethanol is present in an aqueous solution, the fermentation product is feed into a “beer column” which vaporizes the aqueous ethanol solution and produces a gas stream that is approximately 40% ethanol. The gas stream is further distilled to provide a water-ethanol mixture having a concentration of about 95% ethanol. Generally, the final product is produced from a dehydration step in which the remaining 5% water is removed (e.g., using molecular sieves).

There is a continuing need for low cost, efficient processes for providing lignocellulosic ethanol and other biofuels. In particular, the development of new processes that include lignocellulose pretreatment methods that reduce side product formation and waste issues, and which reduce the cost and amount of process equipment required would be beneficial.

### SUMMARY

In some embodiments, the presently disclosed subject matter provides a method of producing an alcohol from a lignocellulosic biomass, the method comprising: providing lignocellulosic biomass; contacting the lignocellulosic biomass with an alkaline composition comprising sodium sulfide and sodium carbonate to provide a pretreated lignocellulosic mixture; contacting the pretreated lignocellulosic mixture with an enzyme composition to provide a fermentable sugar mixture; and fermenting the fermentable sugar mixture to provide an alcohol.

In some embodiments, the lignocellulosic biomass is selected from the group consisting of herbaceous material, agricultural residues, forestry residues, municipal solid wastes, waste paper, pulp and paper mill residues, or a combination thereof. In some embodiments, the lignocellulosic biomass is selected from the group consisting of corn stover, straw, bagasse, miscanthus, sorghum residue, switch grass, bamboo, water hyacinth, hardwood, hardwood chips, softwood chips, hardwood pulp, and softwood pulp.

In some embodiments, contacting the lignocellulosic biomass with an alkaline composition comprises providing a charge comprising total titratable alkali (TTA) ranging from about 4% to about 25%. In some embodiments, contacting the lignocellulosic biomass with an alkaline composition comprises providing a charge comprising TTA ranging from about 12% to about 20%. In some embodiments, the TTA is about 16%.

In some embodiments, the alkaline composition has a sulfidity ranging from about 5% to about 50%. In some embodiments, the sulfidity is about 25%. In some embodiments, the alkaline composition has a pH of between about 8 and about 9.5.

In some embodiments, contacting the lignocellulosic biomass with the alkaline composition takes place at a temperature of between about 100° C. and about 220° C. In some embodiments, the temperature is between about 160° C. and about 170° C.

In some embodiments, contacting the lignocellulosic biomass with the alkaline composition takes place for a period of time between about 0.25 hours and about 4 hours. In some embodiments, contacting the lignocellulosic biomass with the alkaline composition takes place in a carbon steel pressure vessel.

In some embodiments, the method further comprises removing one or more of non-cellulosic material, non-fibrous cellulosic material, and non-degradable cellulosic material from the pretreated lignocellulosic mixture prior to contacting the pretreated lignocellulosic mixture with the enzyme composition. In some embodiments, the removing comprises washing the pretreated lignocellulosic mixture to remove dissolved substances. In some embodiments, the dissolved substances are selected from the group consisting of lignin, degraded cellulose compounds, alkaline chemical compounds, and combinations thereof.

In some embodiments, the lignin is collected and used as an energy source. In some embodiments, the lignin is used as an energy source to provide heat during a distillation or other step during the production of alcohol.

In some embodiments, the alkaline chemical compounds are recycled.

In some embodiments, the removing comprises subjecting the pretreated lignocellulosic mixture to oxygen delignification prior to contacting the pretreated lignocellulosic mixture with the enzyme composition.

In some embodiments, the method further comprises refining solid material in the pretreated lignocellulosic mixture prior to contacting the pretreated lignocellulosic mixture with the enzyme composition, thereby reducing the solid material in size. In some embodiments, the refining provides bundles of cellulose fibers, single cellulose fibers, fragments of single cellulose fibers, or a combination thereof. In some embodiments, the refining is performed using a mechanical refiner.

In some embodiments, the method further comprises subjecting the pretreated lignocellulosic mixture to oxygen delignification and refining solid material in the pretreated lignocellulosic mixture prior to contacting the pretreated lignocellulosic mixture with the enzyme composition.

In some embodiments, the lignocellulosic biomass comprises hardwood chips or softwood chips, and contacting the lignocellulosic biomass with the alkaline composition provides a pretreated lignocellosic mixture comprising about 80% of cellulosic material from the lignocellulosic biomass present as undissolved cellulosic fibers or fragments thereof. In some embodiments, the pretreated lignocellulosic mixture comprises between about 17% and about 24% residual lignin.

In some embodiments, the enzyme composition comprises between about 5 filter-paper units (FPU) and about 40 FPU cellulase per gram of pretreated lignocellulosic material. In some embodiments, the enzyme composition comprises between about 20 FPU and about 40 FPU cellulase per gram of pretreated lignocellulosic material.

In some embodiments, cellulosic material from the pretreated lignocellulosic mixture is contacted with the enzyme composition for a period of time ranging between about 6 hours and about 96 hours. In some embodiments, the period of time ranges between about 40 hours and about 96 hours. In some embodiments, contacting the pretreated lignocellulosic mixture provides a fermentable sugar mixture comprising a combined glucan and xylan yield of between about 50% and about 90% based on total cellulosic material originally present in the lignocellulosic biomass.

In some embodiments, fermenting comprises contacting the fermentable sugar mixture with a microorganism to provide an alcohol mixture and distilling the alcohol mixture to provide the alcohol. In some embodiments, the microorganism is yeast. In some embodiments, the method further comprises dehydrating the alcohol to remove residual water. In some embodiments, the alcohol is ethanol.

In some embodiments, the presently disclosed subject matter provides a composition comprising an alcohol prepared from lignocellulosic biomass using a method that comprises pretreatment of the lignocellulosic biomass with an alkaline solution comprising sodium sulfide and sodium carbonate. In some embodiments, the alcohol is ethanol. In some embodiments, the composition comprises 95% or greater ethanol. In some embodiments, the composition is a fuel mixture comprising ethanol and gasoline.

Accordingly, it is an object of the presently disclosed subject matter to provide a method of producing an alcohol from a lignocellulosic biomass wherein the method comprises contacting the lignocellulosic biomass with an alkaline composition comprising sodium sulfide and sodium carbonate to provide a pretreated lignocellulosic mixture prior to enzymatic hydrolysis and fermentation.

An object of the presently disclosed subject matter having been stated hereinabove, which is addressed in whole or in part by the presently disclosed subject matter, other objects and advantages will become apparent to those of ordinary skill in the art after a study of the following description of the presently disclosed subject matter and in the accompanying non-limiting Examples.

### BRIEF DESCRIPTION OF THE DRAWINGS

FIG. 1 is a block diagram showing a method for converting lignocellulosic biomass into alcohol according to a method of the presently disclosed subject matter.

FIG. 2 is a block diagram showing a method for converting lignocellulosic biomass into alcohol according to a method of the presently disclosed subject matter wherein the method includes an oxygen delignification and/or refining step.

FIG. 3 is a graph showing the effects of total titratable alkali (TTA) on the yield (open squares) and black liquor pH (open diamonds) of green liquor pretreated lignocellulosic biomass. The results are provided as a percentage (%) yield, which refers to the percentage of original biomass remaining after green liquor pretreatment based on weight.

FIG. 4 is a bar graph showing the polysaccharide and lignin content in wood and green liquor (GL) pretreated wood pulps. Polysaccharide and lignin contents (calculated as a percentage of the total content of the pulp) are provided for pulps pretreated with GL having 4% (GL-04), 8% (GL-08), 12% (GL-12), 16% (GL-16), or 20% (GL-20) total titratable alkali (TTA). Glucan content is shown by the darkly shaded portion of the bars, xylan content is shown by the lightly shaded portion of the bars, mannan content is shown by the unshaded portion of the bars, klason lignin content is shown by the striped portion of the bars, acid soluble lignin content is shown by the stippled portion of the bars, and the content of other extractives is shown by the checked portion of the bars.

FIG. 5A is a graph showing the effects of total titratable alkali (TTA) on lignin in green liquor (GL) pretreated pulp. Residual lignin (i.e., the amount of lignin remaining in the pulp following pretreatment, calculated as a percentage of the total mass of the pulp) in pulps pretreated with GL having 4% (GL-04), 8% (GL-08), 12% (GL-12), 16% (GL-16), or 20% (GL-20) TTA are shown. For comparison, the percentage of lignin in non-pretreated wood is also shown (wood). Total yield (i.e., the percentage of original mass recovered following pretreatment) for each GL pretreatment is also indicated (see x-axis).

FIG. 5B is a graph showing the effects of total titratable alkali (TTA) on carbohydrate in green liquor (GL) pretreated pulp. Residual carbohydrate (i.e. the amount of carbohydrates remaining in the pulp following pretreatment, calculated as a percentage of the total mass of the pulp) in pulps pretreated with GL having 4% (GL-04), 8% (GL-08), 12% (GL-12), 16% (GL-16), and 20% (GL-20) TTA are shown. For comparison, the percentage of carbohydrate in non-pretreated wood is also shown (wood). Total yield (i.e., the percentage of original mass recovered following pretreatment) for each GL pretreatment is also indicated (see x-axis).

FIG. 6 is a graph showing how lignin (diamonds) and hemicellulose (xylan, squares; mannan, triangles) removal relates to overall green liquor (GL) pretreatment yield loss. The percentage of lignin, xylan and mannan in the spent GL was calculated and is plotted compared to the percentage of total mass of pulp lost during the pretreatment.

FIG. 7A is a graph showing how total titratable alkali (TTA) of the green liquor (GL) pretreatment and cellulase dosage affect enzymatic hydrolysis efficiency (as measured by percentage weight loss during enzymatic hydrolysis, based on the weight of the GL pretreated pulp prior to enzymatic hydrolysis). Data for pulp pretreated with GL having 4% (GL-04, open diamonds), 8% (GL-08, shaded squares), 12% (GL-12, open diamonds), 16% (GL-16, shaded diamonds), or 20% (open squares) TTA is shown. Cellulase dosage is presented as filter paper units (FPU)/gram pulp.

FIG. 7B is a graph showing how total titratable alkali (TTA) of the green liquor (GL) pretreatment and cellulase dosage affect enzymatic hydrolysis efficiency (as measured by percentage weight loss during enzymatic hydrolysis, based on the weight of the original untreated wood that produced the GL pretreated pulp). Data for pretreatments with GL having 4% (GL-04, open diamonds), 8% (GL-08, shaded squares), 12% (GL-12, open diamonds), 16% (GL-16, shaded diamonds), or 20% (open squares) TTA is shown. Cellulase dosage is presented as filter paper units (FPU)/gram pulp.

FIG. 8 is a graph showing how residual lignin affects enyzmatic hydrolysis at different cellulase dosages (i.e., 5 filter paper units (FPU)/gram pulp, open diamonds; 10 FPU/gram pulp, shaded squares; 20 FPU/gram pulp, open triangles; and 40 FPU/gram pulp, shaded diamonds). Hydrolysis efficiency is measured based on percentage weight loss compared to the weight of the un-pretreated wood used to prepare the pulp for pretreatment and hydrolysis. The percentage of lignin in the pulp following green liquor pretreatment is indicated by the x-axis

FIG. 9A is a graph showing total titratable alkali (TTA) of green liquor (GL) pretreatment and cellulase dosage affect glucan hydrolysis efficiency based on pretreated pulp glucan content. “Glucan, %” refers to the percentage of glucan hydrolyzed based on comparing the remaining glucan content of the hydrolyzed pulp to the glucan content of the GL pretreated pulp. Data for pulp pretreated with GL having 4% (GL-04, open diamonds), 8% (GL-08, shaded squares), 12% (GL-12, open diamonds), 16% (GL-16, shaded diamonds), or 20% (open squares) TTA is shown. Cellulase dosage is presented as filter paper units (FPU)/gram pulp.

FIG. 9B is a graph showing total titratable alkali (TTA) of green liquor (GL) pretreatment and cellulase dosage affect glucan hydrolysis efficiency based on original wood glucan content. “Glucan, %” refers to the percentage of glucan hydrolyzed based on comparing the remaining glucan content of the hydrolyzed pulp to the glucan content of the untreated wood used to prepare the GL pretreated pulp. Data for pulp pretreated with GL having 4% (GL-04, open diamonds), 8% (GL-08, shaded squares), 12% (GL-12, open diamonds), 16% (GL-16, shaded diamonds), or 20% (open squares) TTA is shown. Cellulase dosage is presented as filter paper units (FPU)/gram pulp.

FIG. 10A is a graph showing total titratable alkali (TTA) of green liquor (GL) pretreatment and cellulase dosage affect xylan hydrolysis efficiency based on pretreated pulp xylan content. “Xylan, %” refers to the percentage of xylan hydrolyzed based on comparing the remaining xylan content of the hydrolyzed pulp to the xylan content of the GL pretreated pulp. Data for pulp pretreated with GL having 4% (GL-04, open diamonds), 8% (GL-08, shaded squares), 12% (GL-12, open diamonds), 16% (GL-16, shaded diamonds), or 20% (open squares) TTA is shown. Cellulase dosage is presented as filter paper units (FPU)/gram pulp.

FIG. 10B is a graph showing total titratable alkali (TTA) of green liquor (GL) pretreatment and cellulase dosage affect xylan hydrolysis efficiency based on original wood xylan content. “Xylan, %” refers to the percentage of xylan hydrolyzed based on comparing the remaining xylan content of the hydrolyzed pulp to the xylan content of the untreated wood used to prepare the GL pretreated pulp. Data for pulp pretreated with GL having 4% (GL-04, open diamonds), 8% (GL-08, shaded squares), 12% (GL-12, open diamonds), 16% (GL-16, shaded diamonds), or 20% (open squares) TTA is shown. Cellulase dosage is presented as filter paper units (FPU)/gram pulp.

FIG. 11A is a graph showing total titratable alkali (TTA) of green liquor (GL) pretreatment and cellulase dosage affect combined glucan and xylan hydrolysis efficiency based on pretreated pulp xylan content. “Glucan+Xylan, %” refers to the percentage of combined glucan and xylan hydrolyzed based on comparing the remaining glucan and xylan content of the hydrolyzed pulp to the glucan and xylan content of the GL pretreated pulp. Data for pulp pretreated with GL having 4% (GL-04, open diamonds), 8% (GL-08, shaded squares), 12% (GL-12, open diamonds), 16% (GL-16, shaded diamonds), or 20% (open squares) TTA is shown. Cellulase dosage is presented as filter paper units (FPU)/gram pulp.

FIG. 11B is a graph showing total titratable alkali (TTA) of green liquor (GL) pretreatment and cellulase dosage affect combined glucan and xylan hydrolysis efficiency based on original wood glucan and xylan content. “Glucan+Xylan, %” refers to the percentage of combined glucan and xylan hydrolyzed based on comparing the remaining glucan and xylan content of the hydrolyzed pulp to the glucan and xylan content of the untreated wood used to prepare the GL pretreated pulp. Data for pulp pretreated with GL having 4% (GL-04, open diamonds), 8% (GL-08, shaded squares), 12% (GL-12, open diamonds), 16% (GL-16, shaded diamonds), or 20% (open squares) TTA is shown. Cellulase dosage is presented as filter paper units (FPU)/gram pulp.

FIG. 12A is a graph showing the effect of enzyme hydrolysis time on glucan hydrolysis efficiency in pulps pretreated with green liquor (GL) having 12% (GL-12, open diamonds) or 16% (GL-16, open squares) total titratable alkali (TTA) based on pretreated pulp content. “Glucan, %” refers to the percentage of glucan hydrolyzed based on comparing the remaining glucan content of the hydrolyzed pulp to the glucan content of the GL pretreated pulp.

FIG. 12B is a graph showing the effect of enzyme hydrolysis time on xylan hydrolysis efficiency in pulps pretreated with green liquor (GL) having 12% (GL-12, open diamonds) or 16% (GL-16, open squares) total titratable alkali (TTA) based on pretreated pulp content. “Xylan, %” refers to the percentage of xylan hydrolyzed based on comparing the remaining xylan content of the hydrolyzed pulp to the xylan content of the GL pretreated pulp.

FIG. 13A is a graph showing the effect of enzyme hydrolysis time on glucan hydrolysis efficiency in pulps pretreated with green liquor (GL) having 12% (GL-12, open diamonds) or 16% (GL-16, open squares) total titratable alkali (TTA) based on wood content. “Glucan, %” refers to the percentage of glucan hydrolyzed based on comparing the remaining glucan content of the hydrolyzed pulp to the glucan content of the wood used to prepare the GL pretreated pulp.

FIG. 13B is a graph showing the effect of enzyme hydrolysis time on xylan hydrolysis efficiency in pulps pretreated with green liquor (GL) having 12% (GL-12, open diamonds) or 16% (GL-16, open squares) total titratable alkali (TTA) based on wood content. “Xylan, %” refers to the percentage of xylan hydrolyzed based on comparing the remaining xylan content of the hydrolyzed pulp to the xylan content of the wood used to prepare the GL pretreated pulp.

FIG. 14A is a graph showing the effect of enzyme hydrolysis time on combined glucan and xylan hydrolysis efficiency in pulps pretreated with green liquor (GL) having 12% (GL-12, open diamonds) or 16% (GL-16, open squares) total titratable alkali (TTA) based on pretreated pulp glucan and xylan content. “Glucan+xylan, %” refers to the percentage of combined glucan and xylan hydrolyzed based on comparing the remaining combined glucan and xylan content of the hydrolyzed pulp to the glucan and xylan content of the GL pretreated pulp.

FIG. 14B is a graph showing the effect of enzyme hydrolysis time on combined glucan and xylan hydrolysis efficiency in pulps pretreated with green liquor (GL) having 12% (GL-12, open diamonds) or 16% (GL-16, open squares) total titratable alkali (TTA) based on wood glucan and xylan content. “Glucan+xylan, %” refers to the percentage of combined glucan and xylan hydrolyzed based on comparing the remaining combined glucan and xylan content of the hydrolyzed pulp to the glucan and xylan content of the wood used to prepare the GL pretreated pulp.

### DETAILED DESCRIPTION

The presently disclosed subject matter will now be described more fully hereinafter with reference to the accompanying Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

Unless otherwise defined, all technical and scientific terms used herein have the same meaning as commonly understood by one of ordinary skill in the art to which this presently described subject matter belongs. All publications, patent applications, patents, and other references mentioned herein are incorporated by reference in their entirety.

### I. DEFINITIONS

Following long-standing patent law convention, the terms “a” and “an” mean “one or more” when used in this application, including the claims. Thus, “an enzyme” can refer to a plurality (i.e., two or more) enzymes.

As used herein, the term “about” modifying any amount can refer to the variation in that amount encountered in real world conditions of producing sugars and ethanol, e.g., in the lab, pilot plant, or production facility. For example, the amounts can vary by about 5%, 1%, or 0.5%. Unless otherwise indicated, all numbers expressing quantities of percentage (%), temperature, time, pH, distance, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

The term “and/or” when used to describe two or more activities, conditions, or outcomes refers to situations wherein both of the listed conditions are included or wherein only one of the two listed conditions are included.

The term “comprising”, which is synonymous with “including,” “containing,” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic_and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

The term “saccharide” refers to a carbohydrate monomer, oligomer or larger polymer. Thus, a saccharide can be a compound that includes one or more cyclized monomer unit based upon an open chain form of a compound having the chemical structure H(CHOH)ₙC(═O)(CHOH)ₘH, wherein the sum of n+m is an integer between 2 and 8. Thus, the monomer units can include trioses, tetroses, pentoses, hexoses, heptoses, nonoses, and mixtures thereof. In some embodiments, each cyclized monomer unit is based on a compound having a chemical structure wherein n+m is 4 or 5. Thus, saccharides can include monosaccharides including, but not limited to, aldohexoses, aldopentoses, ketohexoses, and ketopentoses such as arabinose, lyxose, ribose, xylose, ribulose, xylulose, allose, altrose, galactose, glucose, gulose, idose, mannose, talose, fructose, psicose, sorbose, and tagatose, and to hetero- and homopolymers thereof. Saccharides can also include disaccharides including, but not limited to sucrose, maltose, lactose, trehalose, and cellobiose, as well as hetero- and homopolymers thereof.

The term “oligosaccharide” refers to polysaccharides having a degree of polymerization of between about 2 and about 10.

The terms “fermentable sugar” and “sugar” can be used interchangeably and refer to oligosaccharides, monosaccharides and mixtures thereof that can be used as a carbon source in a fermentation process. Fermentable monosaccharides include arabinose, glyceraldehyde, dihydroxyacetone, erythrose, ribose, ribulose, xylose, glucose, galactose, mannose, fucose, fructose, sedoheptulose, neuraminic acid, or mixtures of these. Fermentable disaccharides include sucrose, lactose, maltose, gentiobiose, or mixtures thereof. “Sugar” can also be used to refer to polysaccharides that require further enzymatic treatment prior to fermentation.

The term “lignocellulosic” refers to a composition comprising both lignin and cellulose. In some embodiments, lignocellulosic material can comprise hemicellulose, a polysaccharide which can comprise saccharide monomers other than glucose. Typically, lignocellulosic materials comprise between about 38-50% cellulose, 15-30% lignin, and 23-32% hemicellulose.

Lignocellulosic biomass include a variety of plants and plant materials, such as, but not limited to, papermaking sludge; wood, and wood-related materials, e.g., saw dust, or particle board, leaves, or trees, such as poplar trees; grasses, such as switchgrass and sudangrass; grass clippings; rice hulls; bagasse (e.g., sugar cane bagasse), jute; hemp; flax; bamboo; sisal; abaca; hays; straws; corn cobs; corn stover; whole plant corn, and coconut hair. In some embodiments, lignocellulosic biomass is selected from the group including, but not limited to, herbaceous material, agricultural residues, forestry residues, municipal solid wastes, waste paper, pulp and paper mill residues, or a combination thereof. In some embodiments, lignocellulosic biomass is selected from the group including, but not limited to, corn stover, straw, bagasse, miscanthus, sorghum residue, switch grass, bamboo, water hyacinth, hardwood, hardwood, softwood, wood chips, and wood pulp.

“Lignin” is a polyphenolic material comprised of phenyl propane units linked by ether and carbon-carbon bonds. Lignins can be highly branched and can also be crosslinked. Lignins can have significant structural variation that depends, at least in part, on the plant source involved.

The term “glucan” refers to a polysaccharide comprising glucose monomers linked by glycosidic bonds.

The term “cellulose” refers to a polysaccharide of β-glucose (i.e., β-1,4-glucan) comprising β-(1-4) glycosidic bonds. The term “cellulosic” refers to a composition comprising cellulose.

The term “hemicellulose” refers to polysaccharides comprising sugars other than glucose. Hemicelluloses can comprise a single type of non-glucose sugar monomer (e.g., zylose, mannose, etc.) or monomers of two or more different sugars. Thus, xylan (polymerized xylose) and mannan (polymerized mannose) are exemplary hemicelluloses. Hemicellulose can be highly branched. Hemicellulose can be chemically bonded to lignin and can further be randomly acetylated, which can reduce enzymatic hydrolysis of the glycosidic bonds in hemicellulose.

The terms “glycosidic bond” and “glycosidic linkage” refer to a linkage between the hemiacetal group of one saccharide unit and the hydroxyl group of another saccharide unit.

The term “biofuel” refers to a fuel that is derived from biomass, i.e., a living or recently living biological organism, such as a plant or an animal waste.

Biofuels include, but are not limited to, biodisel, biohydrogen, biogas, biomass-derived dimethylfuran (DMF), and the like. In particular, the term “biofuel” can be used to refer to biomass-derived alcohols (e.g., bioalcohol), such as ethanol, methanol, propanol, or butanol, which can be denatured, if desired prior to use. The term “biofuel” can also be used to refer to fuel mixtures comprising biomass-derived fuels, such as alcohol/gasoline mixtures (i.e., gasohols). Gasohols can comprise any desired percentage of biomass-derived alcohol (i.e., about 10%, 15%, 20%, 25%, 30%, 35%, 40%, 45%, 50%, 55%, 60%, 65%, 70%, 75%, 80%, 85%, 90%, or 95% biomass-derived alcohol). For example, one useful biofuel-based mixture is E85, which comprises 85% ethanol and 15% gasoline.

The term “pretreat” generally refers to a chemical, microbial, or mechanical method of treating biomass to make it more amendable to enzymatic hydrolysis and/or microbial fermentation. For example, pretreating can relate to removing or altering lignin, removing hemicellulose, decrystallizing cellulose, removing acetyl groups (e.g., through chemical or enzymatic hydrolysis of the acetyl ester), reducing the degree of polymerization of cellulose (i.e., hydrolysis of glycosidic bonds), expanding the structure of the lignocellulosic material to increase pore volume and internal surface area.

The term “green liquor” refers to an alkaline composition, such as that used in alkaline pulping during paper production, comprising sodium sulfide (Na₂S) and sodium carbonate(Na₂CO₃). In some embodiments, green liquor can further comprise sodium sulfate (Na₂SO₄).

The term “total titratable alkali” refers to the weight percentage of combined alkali species (e.g., Na₂CO₃, Na₂S, and NaOH) in a solution, expressed as Na₂O.

The term “sulfidity” refers to the weight percentage of alkaline sulfur compounds in a solution compared to the total titratable alkali.

The term “delignification” refers to the removal of some or all of the lignan present in a lignin-containing sample. Delignification can be performed via chemical, mechanical, or enzymatic processes or combinations thereof.

“Oxygen delignification” refers to a delignification process wherein biomass (e.g., green liquor pretreated biomass) is contacted with oxygen gas in a pressurized vessel at an elevated temperature in an alkaline environment. Oxygen delignification is used in the paper industry to treat paper pulp in part to reduce the consumption of bleaching chemicals.

The term “refining” refers to a mechanical process of treating lignocellulosic-containing solids in order to beat, bruise, cut, and/or fibrillate the fibers therein. Thus, refining can be used to reduce lignocellulosic-containing solids in size as well as to providing material comprising bundles of cellulosic fibers, separate cellulosic fibers, fragments of cellulosic fibers, and combinations thereof.

The term “enzyme” refers to a protein that catalyzes the conversion of one molecule into another. The term “enzyme” as used herein includes any enzyme that can catalyze the transformation of a biomass-derived molecule to another biomass-derived molecule. In particular, enzymes include those which can degrade or transform saccharide, cellulose, or lignocellulose molecules to provide fermentable sugars and alcohols.

The terms “lignocellulytic enzyme” “lignocellulose-processing enzyme”, and “lingocellulose-hydrolyzing enzyme” refer to enzymes that are involved in the disruption and/or degradation of lignocellulose. The disruption of lignocellulose by lignocellulytic enzymes leads to the formation of substances including monosaccharides, disaccharides, polysaccharides and phenols. Lignocellulytic enzymes include, but are not limited to, cellulases, hemicellulases and ligninases. Thus, lignocellulytic enzymes include saccharification enzymes, i.e., enzymes which hydrolyze (i.e., depolymerize) polysaccharides. Saccharification enzymes and their use in biomass treatments have been previously reviewed. See Lynd, L. R., et al., Microbiol. Mol. Biol. Rev., 66, 506-577 (2002).

The term “cellulase” when used generally can refer to enzymes involved in cellulose degradation. Cellulase enzymes are classified on the basis of their mode of action. There are two basic kinds of cellulases: the endocellulases, which cleave polysaccharide polymer chains internally; and the exocellulases, which cleave from the reducing and non-reducing ends of molecules generated by the action of endocellulases. Cellulases include cellobiohydrolases, endoglucanases, and β-D-glucosidases. Endoglucanases randomly attack the amorphous regions of cellulose substrates, yielding mainly higher oligomers. Cellulobiohydrolases are exocellulases which hydrolyze crystalline cellulose and release cellobiose (glucose dimer). Both types of enzymes hydrolyze-1,4-glycosidic bonds. β-D-glucosidases or cellulobiase converts oligosaccharides and cellubiose to glucose.

As used herein, the term “cellulase” is typically used more specifically to refer to the enzyme is cellulase (E.C. 3.2.1.4), also known as an endoglucanase, which catalyzes the hydrolysis of 1,4-β-D-glycosidic linkages. The cellulase can be of microbial origin, such as derivable from a strain of a filamentous fungus (e.g., Aspergillus, Trichoderma, Humicola, Fusarium). Commercially available cellulase preparations which can be used include, but are not limited to, CELLUCLAST™, CELLUZYME™, CEREFLO™, and ULTRAFLO™ (available from Novozymes AIS, Bagsvaerd, Denmark), SPEZYME™ CE and SPEZYME™ CP (available from Genencor International, Inc., Rochester, N.Y., United States of America) and ROHAMENT® CL (available from AB Enzymes GmbH, Darmstadt, Germany).

Hemicellulases are enzymes that are involved in hemicellulose degradation. Hemicellulases include xylanases, arabinofuranosidases, acetyl xylan esterases, glucuronidases, mannanases, galactanases, and arabinases. Similar to cellulase enzymes, hemicellulases are classified on the basis of their mode of action: the endo-acting hemicellulases attack internal bonds within the polysaccharide chain; the exo-acting hemicellulases act progressively from either the reducing or non-reducing end of polysaccharide chains. More particularly, endo-acting hemicellulases include, but are not limited to, endoarabinanase, endoarabinogalactanase, endoglucanase, endomannanase, endoxylanase, and feraxan endoxylanase. Examples of exo-acting hemicellulases include, but are not limited to, α-L-arabinosidase, β-L-arabinosidase, α-1,2-L-fucosidase, α-D-galactosidase, β-D-galactosidase, β-D-glucosidase, β-D-glucuronidase, β-D-mannosidase, β-D-xylosidase, exo-glucosidase, exo-cellobiohydrolase, exo-mannobiohydrolase, exo-mannanase, exo-xylanase, xylan α-glucuronidase, and coniferin β-glucosidase.

Ligninases are enzymes that are involved in the degradation of lignin. A variety of fungi and bacteria produce ligninases. Lignin-degrading enzymes include, but are not limited to, lignin peroxidases, manganese-dependent peroxidases, hybrid peroxidases (which exhibit combined properties of lignin peroxidases and manganese-dependent peroxidases), and laccases. Hydrogen peroxide, required as a co-substrate by the peroxidases, can be generated by glucose oxidase, aryl alcohol oxidase, and/or lignin peroxidase-activated glyoxal oxidase.

The term “filter paper unit” (or FPU) refers to the amount of enzyme required to liberate 2 mg of reducing sugar (e.g., glucose) from a 50 mg piece of Whatman No. 1 filter paper in 1 hour at 50° C. at approximately pH 4.8.

The terms “hydrolyze,” and variations thereof refer to the process of converting polysaccharides (e.g., cellulose) to fermentable sugars, e.g., through the hydrolysis of glycosidic bonds. This process can also be referred to as saccarification. Hydrolysis can be effected with enzymes or chemicals. Enzymes can be added to biomass directly (e.g., as a solid or liquid enzyme additive) or can be produced in situ by microbes (e.g., yeasts, fungi, bacteria, etc.). Hydrolysis products include, for example, fermentable sugars, such as glucose and other small (low molecular weight) oligosaccharides such as monosaccharides, disaccharides, and trisaccharides. Hydrolysis products can also simply include lower molecular weight polysaccharides than those in the original cellulose or lignocellulose. “Suitable conditions” for saccharification refer to various conditions including pH, temperature, biomass composition, and enzyme composition.

“Fermentation” or “fermenting” can refer to the process of transforming an organic molecule into another molecule using a micro-organism. For example, “fermentation” can refer to transforming sugars or other molecules from biomass to produce alcohols (e.g., ethanol, methanol, butanol); organic acids (e.g., citric acid, acetic acid, itaconic acid, lactic acid, gluconic acid); ketones (e.g., acetone), amino acids (e.g., glutamic acid); gases (e.g., H₂ and CO₂), antibiotics (e.g., penicillin and tetracycline); enzymes; vitamins (e.g., riboflavin, B₁₂, beta-carotene); and/or hormones. Thus, fermentation includes alcohol fermentation. Fermentation also includes anaerobic fermentations.

Fermenting can be accomplished by any organism suitable for use in a desired fermentation step, including, but not limited to, bacteria, fungi, archaea, and protists. Suitable fermenting organisms include those that can convert mono-, di-, and trisaccharides, especially glucose and maltose, or any other biomass-derived molecule, directly or indirectly to the desired fermentation product (e.g., ethanol, butanol, etc.). Suitable fermenting organisms also include those which can convert non-sugar molecules to desired fermentation products.

In some embodiments, the fermenting is effected by a fungal organism (e.g., yeast or filamentous fungi). The yeast can include strains from a Pichia or Saccharomyces species. In some embodiments, the yeast can be Saccharomyces cerevisiae. In some embodiments, the fermenting is effected by bacteria. For example, the bacteria can be Clostridium acetobutylicum (e.g., when butanol is the desired fermentation product) or Corynebacterium glutamicum (e.g., when monosodium glutamate (MSG) is the desired fermentation product). In some embodiments, the micro-organism (e.g. yeast or bacteria) can be a genetically modified micro-organism. In some instances, the organism can be yeast or other organism having or modified to be active in the presence of high concentrations of alcohol.

Thus “fermentation” and grammatical variations thereof refer to the conversion of a fermentable sugar to an alcohol (e.g., methanol, ethanol, propanol, butanol, etc.). The particular product of a given alcohol fermentation can be determined by the biocatalyst used in the fermentation and/or the substrate of fermentation (i.e., the type of fermentable sugar being converted).

In certain embodiments, fermenting can comprise contacting a mixture including biomass-derived sugars with an alcohol-producing biocatalyst, such as a yeast or another alcohol-producing microbe. In some embodiments, fermenting involves simultaneous saccharification (e.g., hydrolysis) and fermentation (SSF). The amount of fermentation biocatalyst employed can be selected to effectively produce a desired amount of ethanol in a suitable time and/or upon the sugar content of a given fermentation mixture. The use of alcohol-producing biocatalyst can increase the rate of saccharification by reducing the concentration of sugars, which can inhibit saccharification biocatalysts.

“Suitable conditions” for alcohol fermentation can refer to conditions that support the production of ethanol or another alcohol by a biocatalyst. Such conditions can include pH, nutrients, temperature, atmosphere, and other factors.

“Dehydrating” refers to removing the residual water left in ethanol following distillation. The residual water is generally about 5% by volume. Dehydration can be performed using molecular sieves.

### II. REPRESENTATIVE METHODS OF THE PRESENTLY DISCLOSED SUBJECT MATTER

The kraft (or sulfate) process used in paper plants during pulping (i.e., the conversion of wood into wood pulp) refers to a process of treating wood chips with a mixture of sodium hydroxide and sodium sulfide (i.e., “white liquor”) to remove lignin. During the process, sulfide (S²) and/or hydrosulfide (HS) ions cleave ether linkages present in lignin. The wood chips and white liquor are typically reacted for several hours at temperatures between about 130-180° C. The hemicellulose and lignin present in the wood chips degrade into fragments that are soluble in strong alkaline solutions. The remaining solid pulp is collected and washed, while the liquids (known as “black liquor”), which contain lignin, degraded hemicellulose and cellulose fragments, sodium carbonate, sodium sulfate and other inorganic salts are also collected. In order to keep the pulping process economically favorable, the inorganic chemicals in the black liquor are recovered and recycled by first burning the black liquor in a recovery boiler. During the burning, sodium sulfate is converted to sodium sulfide. The residue from the recovery boiler is then dissolved in water, providing a solution of sodium carbonate and sodium sulfide (i.e., “green liquor”). The green liquor is treated with calcium oxide to regenerate white liquor and a calcium carbonate precipitate, which is subsequently converted to lime (i.e., calcium oxide).

The presently disclosed subject matter provides in some embodiments a method of producing alcohol from lignocellulosic biomass using a “green liquor” pretreatment, wherein lignocellulosic biomass is contacted with an alkaline solution comprising sodium sulfide and sodium carbonate. While some alkaline pretreatments (e.g., sodium hydroxide, lime, ammonia, sodium hydroxide/ozone combinations) have been used to treat lignocellulosic materials as part of biomass-to-ethanol processes prior to the presently disclosed subject matter, alkaline pretreatments are generally disfavored because they can degrade wood to provide products that cannot be fermented. Further, prior to the presently disclosed subject matter, green liquor pretreatment would appear to have little capability to remove lignin from the biomass matrix, leaving high lignin content fibers that would be difficult to process with cellulase enzymes. However, as described herein, it has been unexpectedly found that significant alkaline degradation during green liquor pretreatments of lignocellulosic biomass can be avoided by selection of conditions that preserve cellulose present in the original wood, while still opening up the wood structure so that hydrolysis enzymes can penetrate the fibers sufficiently to hydrolyze cellulose to fermentable sugars.

In addition, the presently disclosed subject matter provides methods that can allow for recovery of lignin from the biomass, which can be re-used as a fuel. In some embodiments, the reuse of lignin can reduce or eliminate the need for purchasing additional fuel to provide power for the biomass-to-alcohol method. Lignin has about 2 times the fuel value of cellulose. Thus, in some embodiments of the presently disclosed subject matter, lignin is recovered at high solids content so that it is available for steam production. In some embodiments, spent liquor from the green liquor pretreatment step (e.g., liquor that comprises dissolved organic material and inorganic chemicals) is recovered. This “black liquor” can be evaporated and concentrated to about 70% solids. In addition, lignin recovered after enzyme hydrolysis of the pretreated biomass can be added to the concentrated black liquor to provide a mixture that can sustain combustion.

In some embodiments, systems for performing the presently disclosed methods can be configured by repurposing the components of a pulping mill that previously used the kraft pulping process. Such repurposing can allow for the employment of the presently disclosed methods with relatively low capitol investment compared to many other proposed biomass-to-ethanol methods. In particular, the repurposing of paper mills can make use of recovery boilers previously used to burn pulping “black liquors.”

In some embodiments, the presently disclosed subject matter provides a method of producing an alcohol from a lignocellulosic biomass, wherein the method can comprise: providing lignocellulosic biomass; contacting the lignocellulosic biomass with an alkaline composition comprising sodium sulfide and sodium carbonate to provide a pretreated lignocellulosic mixture; contacting the pretreated lignocellulosic mixture with an enzyme composition to provide a fermentable sugar mixture; and fermenting the fermentable sugar mixture to provide an alcohol. Any suitable (e.g., inexpensive and/or readily available) type of biomass can be used. Suitable lignocellulosic biomass for use in the presently disclosed methods can be selected from the group comprising, but not limited to, herbaceous material, agricultural residues, forestry residues, municipal solid wastes, waste paper, pulp and paper mill residues, and combinations thereof. Thus, in some embodiments, the biomass can comprise, for example, corn stover, straw, bagasse, miscanthus, sorghum residue, switch grass, bamboo, water hyacinth, hardwood, hardwood chips, softwood chips, hardwood pulp, and/or softwood pulp, or the like. In some embodiments, the biomass can be chosen based upon a consideration such as, but not limited to, cellulose and/or hemicellulose content, lignin content, growing time/season, growing location/transportation costs, growing costs (e.g., fertilizer and or irrigation requirements), harvesting costs, and the like.

Prior to pretreatment with green liquor, the biomass can be washed and/or reduced in size (e.g., by chopping or crushing) to a convenient size (e.g., to aid in moving the biomass or in mixing the biomass with the green liquor). Thus, in some embodiments, providing biomass can comprise harvesting a lignocellulose-containing plant (e.g., a hardwood or softwood tree), chopping the tree into wood chips, and washing either the tree or the cut chips, (e.g., to remove any residual soil).

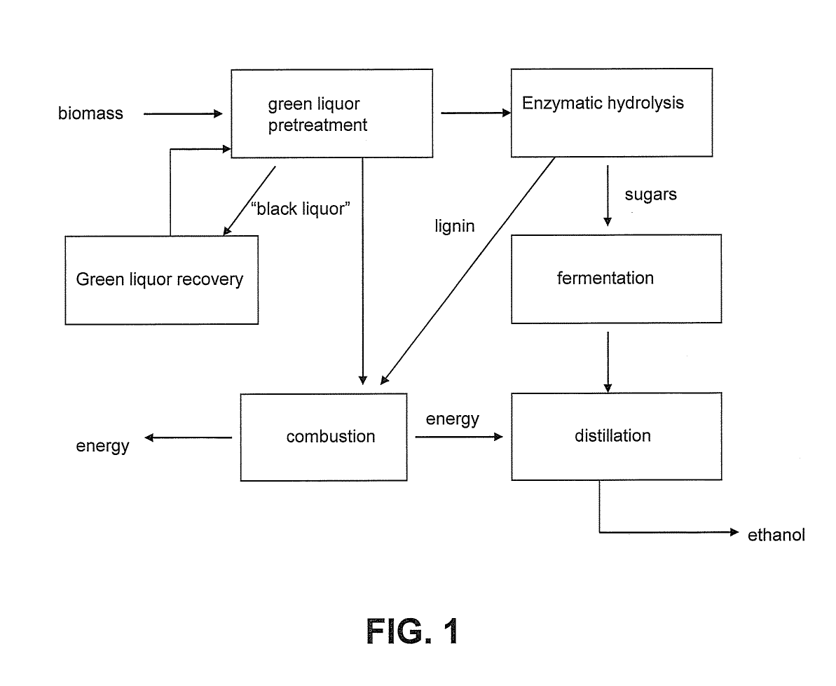

As shown in FIG. 1, in one representative, non-limiting embodiment, the presently disclosed method can be initiated by providing lignocellulosic biomass and introducing the biomass into a pressure vessel wherein green liquor pretreatment can take place. Unlike in the “dilute acid” process described above, the pressure vessel used in the presently disclosed methods can optionally be a lower cost pressure vessel made of, for example, carbon steel. Suitable pressure vessels include, but are not limited to the “PANDIA™ Digester” (Voest-Alpine Industrieanlagenbau GmbH, Linz, Austria), the “DEFIBRATOR™ Digester” (Sunds Defibrator AB Corporation, Stockholm, Sweden) and the “KAMYR™ Digester” (Andritz, Inc., Glens Falls, N.Y., United States of America). Green liquor is added, along with steam, to the pressure vessel. The contents can then be kept at a temperature of between 100-220° C. (e.g., about 100, 110, 120, 130, 140, 150, 160, 170, 180, 190, 200, 210, or 220° C.) for a period of time. In some embodiments, the contents are kept at a temperature of between about 100-180° C. for a period of time. In some embodiments, the contents are held at a temperature of about 160-170° C. for a period of time. The period of time can be from about 0.25 to 4.0 hours (e.g., about 0.25, 0.5, 0.75, 1.0, 1.25, 1.5, 1.75, 2.0, 2.25, 2.5, 2.75, 3.0, 3.25, 3.5, 3.75, or 4 hours), after which the “cooked” contents of the digester are discharged. In some embodiments, the period of time is from about 0.5 hours to about 2.0 hours.

In some embodiments, the lignocellulosic biomass is contacted with an alkaline composition such that the contacting provides a chemical charge comprising total titratable alkali (TTA) ranging from about 4% to about 25% (e.g., about 4%, 6%, 8%, 10%, 12%, 14%, 16%, 18%, 20%, 22%, 24%, or 25%). In some embodiments, the TTA ranges between about 12% and about 20%. In some embodiments, the TTA is about 16%. In some embodiments, the alkaline composition has a sulfidity ranging from about 5% to about 50%. In some embodiments, the sulfidity ranges between about 12% and about 37% (e.g., about 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36% or 37%). In some embodiments, the sulfidity is about 25%. In some embodiments, the alkaline composition has a pH of between about 8 and about 9.5 (e.g., about 8.0, 8.1, 8.2, 8.3, 8.4, 8.5, 8.6, 8.7, 8.8, 8.9, 9.0, 9.1, 9.2, 9.3, 9.4, or 9.5), depending on TTA.

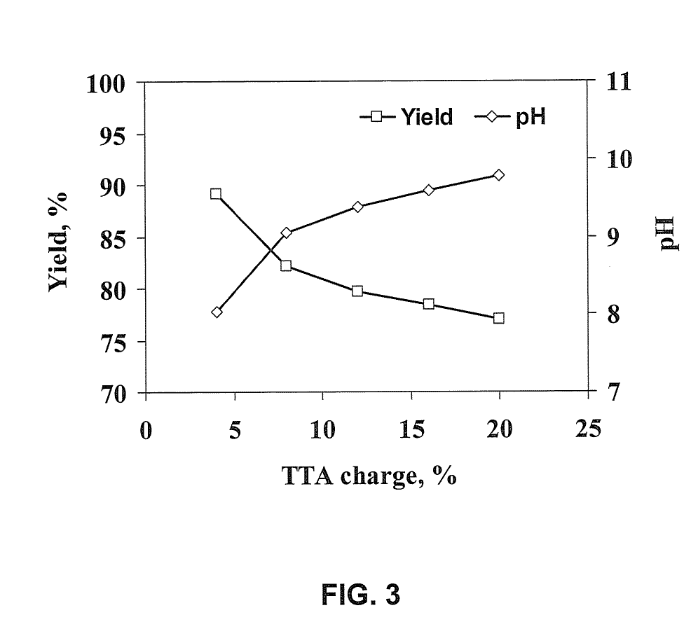

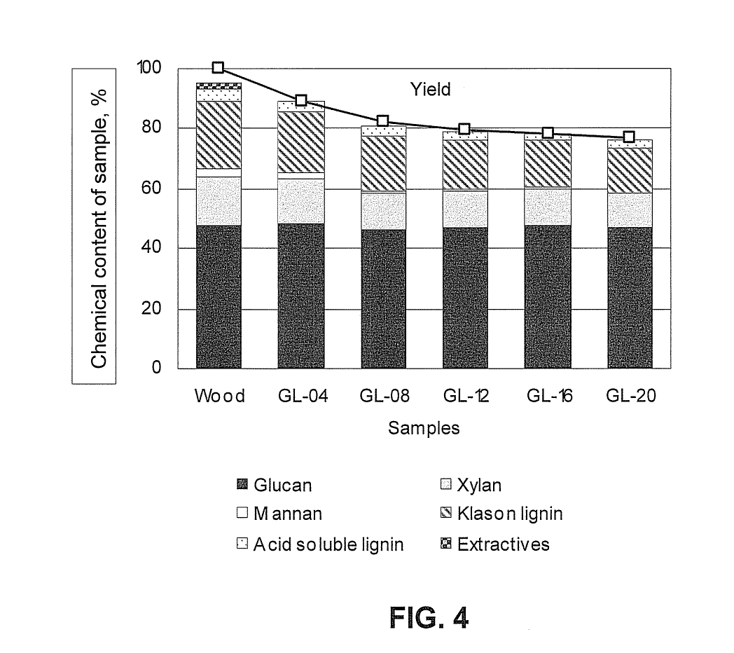

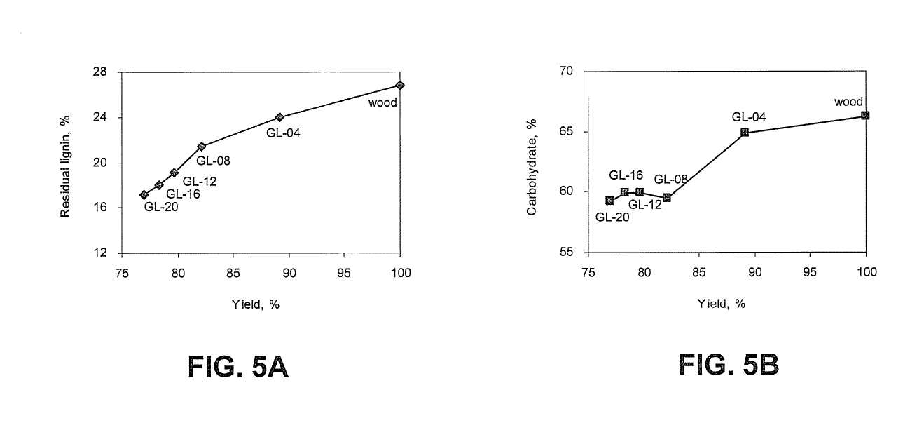

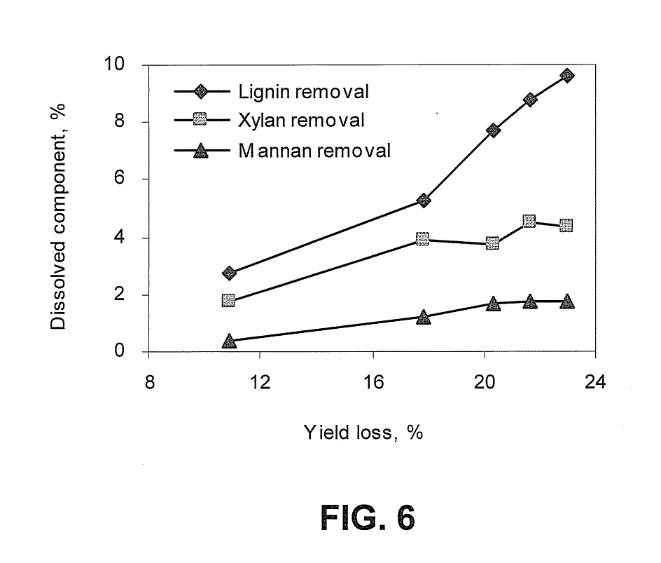

In the pretreatment step, approximately 10-23% of the biomass can be dissolved by the green liquor. If lignin and cellulosic and hemicellulosic materials are present in the original wood in proportions of approximately 1:3 (i.e., 75% cellulose and hemicellulose), the cooked chips and the dissolved matter can have roughly the same proportions. Stated another way, if the yield of pulp is about 80% of the original wood, it can contain about 60 parts original cellulose and about 20 parts original lignin. While some of the original cellulose can be lost, the loss can allow for later penetration of the remaining cellulose with enzymes (e.g., cellulose or another lignocellulose-degrading enzyme). Without the loss during pretreatment, enzymatic hydrolysis can be inhibited. As shown in FIG. 3, depending on the TTA of the green liquor, between about 77-90% of the original biomass content is recovered following the green liquor pretreatment. In some embodiments, green liquor pretreatment (e.g., of hardwood or softwood chips) leaves about 80% of the original cellulosic material as undissolved solids, such as undissolved cellulosic fibers or fragments thereof. The components of the material recovered following green liquor pretreatment are shown by chemical type in FIGS. 4, 5A and 5B. FIG. 6 shows the percentage of original lignin, xylan, and mannan removed from biomass during green liquor pretreatment. The removed lignin, xylan and mannan are dissolved in the spent green liquor (i.e., the black liquor).

In some embodiments, the pretreatment can further comprise the use of one or more additives to increase the yield (i.e., the non-removal during green liquor pretreatment) of carbohydrates (e.g., cellulose and hemicellulose). Such additives include, but are not limited to, anthraquinone and sodium polysulfides. In some embodiments, the green liquor can further comprise one or more additives.

Green liquor pretreated biomass (e.g., biomass chips) can be refined using any suitable mechanical refining device to further break down the material in size prior to enzymatic hydrolysis. For example, the contents of the pretreatment pressure vessel can be discharged into a mechanical disc refiner or PFI refiner (or other refiner typically used in the pulping industry) to break the cooked (i.e., pretreated) chips open and reduce the cooked chips to form small pieces. In some embodiments, the refining can provide bundles of cellulose fibers, single cellulose fibers, fragments of cellulose fibers, or combinations thereof. In some embodiments, refining provides largely single fibers and bundles of single fibers. In some embodiments, refining can provide pretreated biomass wherein over 90% of the material is single fibers or fragments of single fibers.

In some embodiments, the cooked biomass can be treated to remove one or more of non-cellulosic material, non-fibrous cellulosic material, and non-degradable cellulosic material prior to enzymatic hydrolysis. For example, the cooked fibers can be washed with water to remove dissolved substances, including degraded, but non-fermentable cellulose compounds, solubilized lignin, and/or any remaining alkaline chemicals that were used for cooking. In some embodiments, these materials can be reused. For example, black liquor and any wash water can be combined and concentrated to a burnable lignin-containing solution that can be used as an energy source in an optional combustion step. See FIG. 1. In some embodiments, lignin collected following an enzymatic hydrolysis step can be optionally added to the black liquor, as well, to increase lignin content. See FIG. 1. In some embodiments, the lignin can be used, for example, to provide heat during the distillation of alcohol or another step in the biomass-to-alcohol process. In some embodiments, the alkaline chemicals can be recycled, e.g., by processes known in the pulping art, in an optional green liquor recovery step. See FIG. 1.

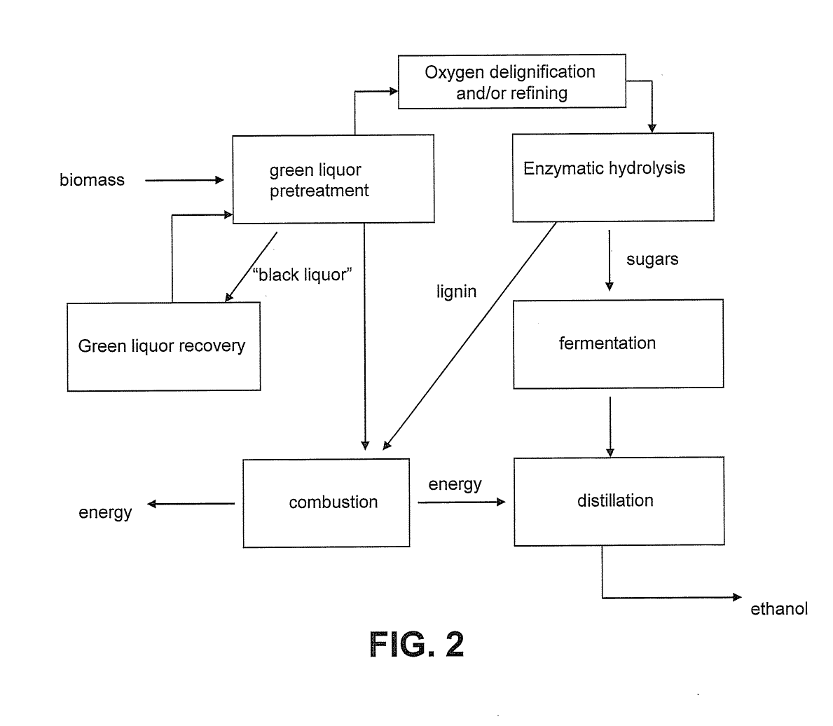

Generally, not all of the lignin is removed by green liquor pretreatment. In some embodiments, the pretreated lignocellulosic material contains between about 17% and about 24% residual lignin. See FIG. 5A. In some embodiments, at least a portion of the residual lignin can be removed from the green liquor treated biomass by oxygen delingnification. Accordingly, in some embodiments, solids from the pretreated lignocellulosic mixture can be collected (e.g., via filtration or decanting of any liquids), dried (e.g., in an oven) and placed in an aqueous alkaline solution (e.g., water comprising 2-5% NaOH). The alkaline solution of solids can then be placed in a pressurized vessel and treated with oxygen gas at an elevated temperature, such as between about 60° C. and about 150° C. (e.g., about 60, 65, 70, 75, 80, 85, 90, 95, 100, 105, 110, 115, 120, 125, 130, 135, 140, 145, or 150° C.), for a period of time, such as between about 10 minutes and about 4 hours (e.g., about 10 minutes, 20 minutes, 30 minutes, 40 minutes, 50 minutes, 1 hour, 1.25 hours, 1.5 hours, 1.75 hours, 2 hours, 2.25 hours, 2.5 hours, 2.75 hours, 3 hours, 3.25 hours, 3.5 hours, 3.75 hours, or 4 hours). In some embodiments, the elevated temperature is about 110° C. In some embodiments, the period of time is about 1 hour. This treatment can oxidize lignin present in the solids. The oxidized lignin can then be removed via washing (e.g., in water). In some embodiments, oxygen delignification can be performed prior to a refining step, such that the final pretreated lignocellulosic biomass mixture (i.e., the biomass used for enzymatic hydrolysis and fermentation) is a mixture that has been treated with green liquor, subjected to oxygen delignification, and refined. See FIG. 2.

Referring again to FIGS. 1 and 2, following green liquor pretreatment and/or any other desired pretreatment steps (e.g., washing, drying, refining, delignifying), the pretreated chips and/or fibers can then be subjected to enzymatic hydrolysis for conversion to fermentable sugars (e.g., glucose). In some embodiments, the enzymatic hydrolysis is performed using cellulase. In some embodiments, between about 5 filter paper units (FPU) and about 85 FPU are used per gram of pretreated lignocellulosic biomass. In some embodiments, between about 5 and about 40 FPU/gram pretreated biomass are used (e.g., about 5, 10, 15, 20, 25, 30, 35, or 40 FPU/gram). In some embodiments, between about 20 FPU and about 40 FPU/gram are used. Other types of enzymes can also be used, both to break down cellulose or to breakdown remaining hemicellulose or lignin. Thus, in some embodiments, the enyzmatic hydrolysis is performed using cellulase in combination with one or more additional enzymes (e.g., xylanase, β-glucosidase, etc.). The enzymes can be added all at once, or portion-wise, during the hydrolysis.

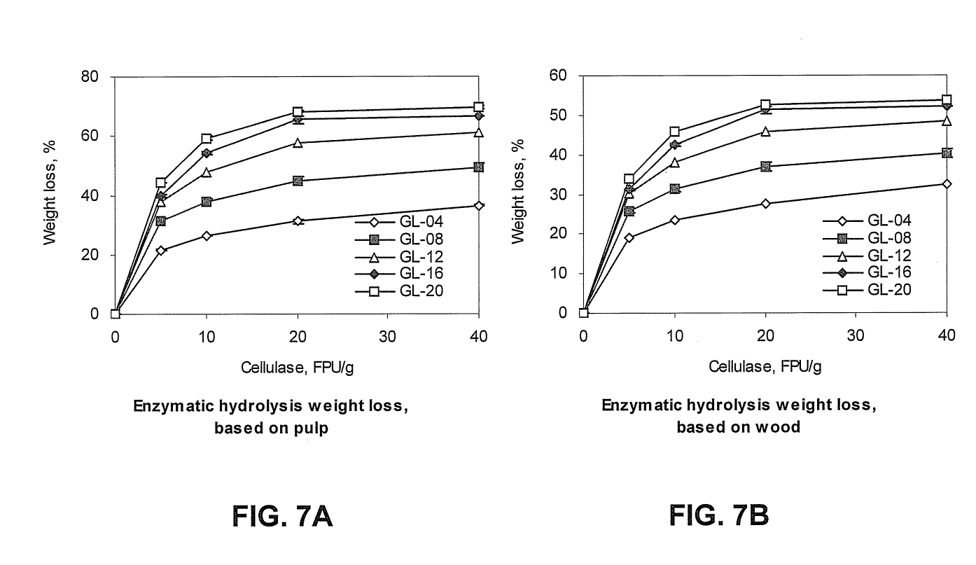

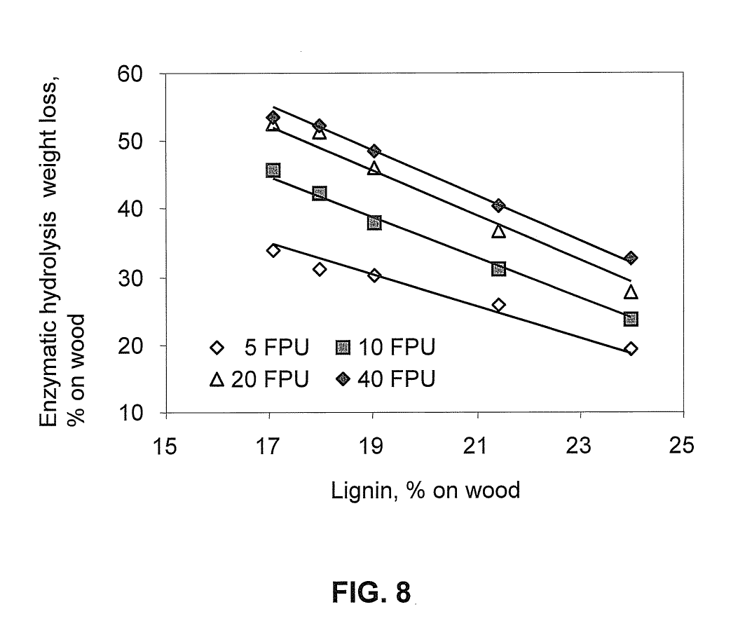

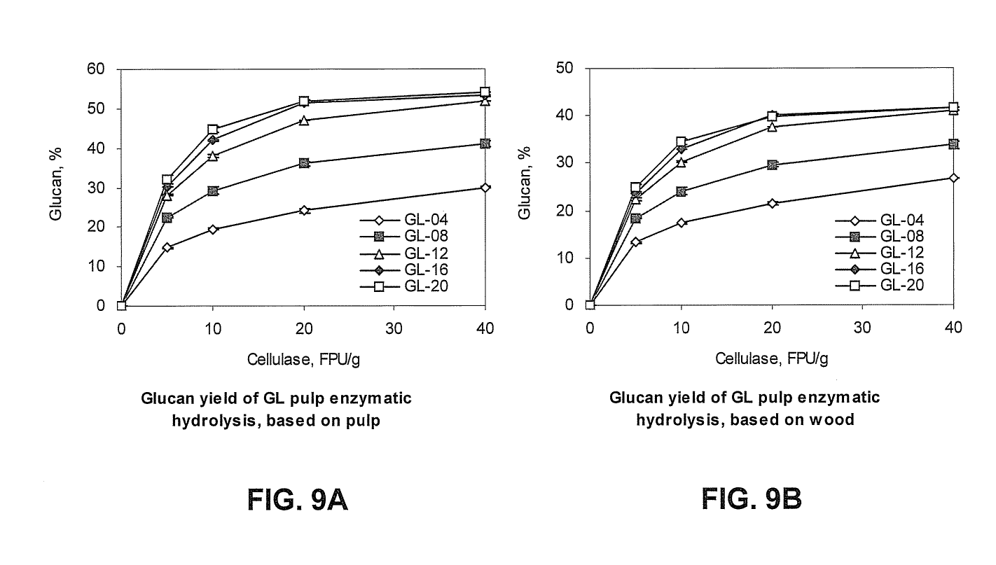

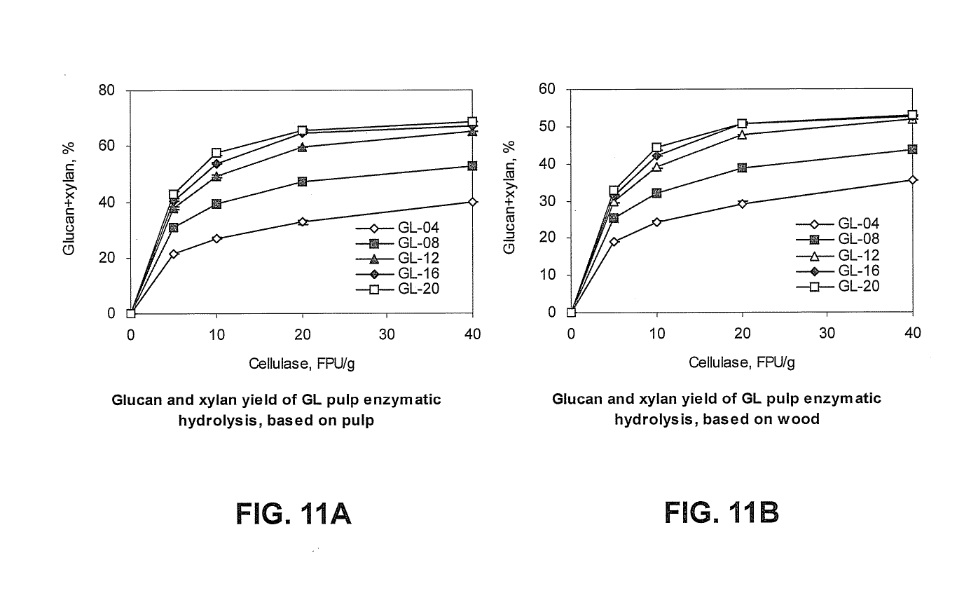

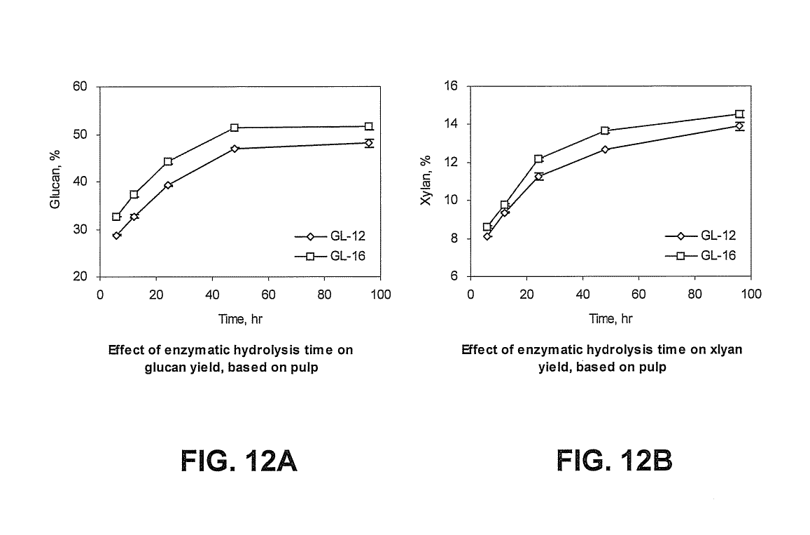

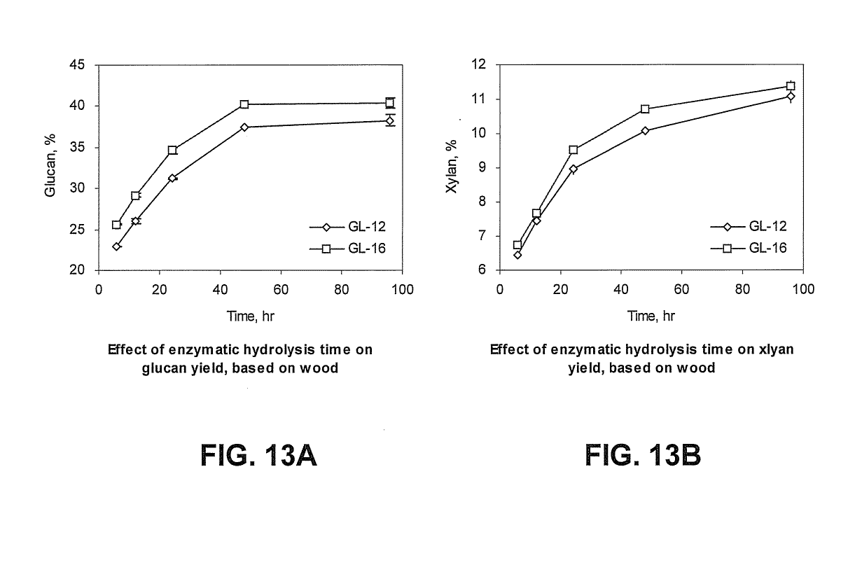

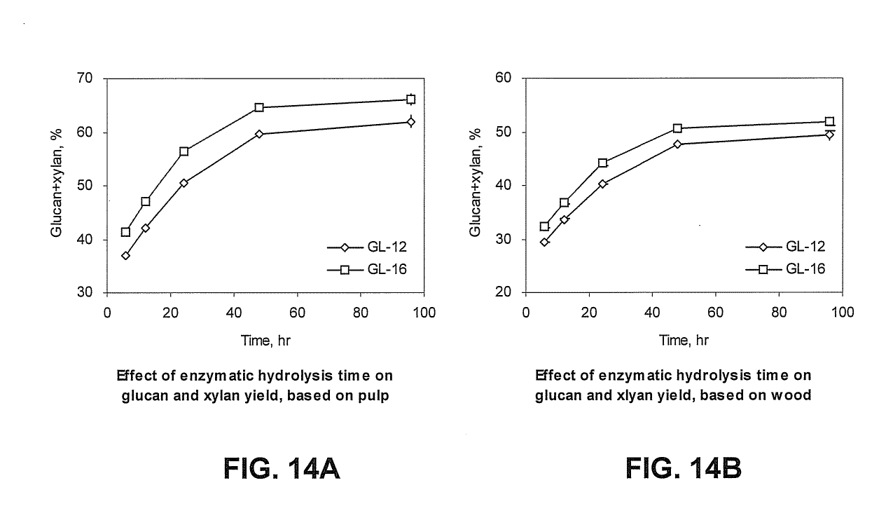

According to the presently disclosed subject matter, the green liquor pretreatment allows for conversion of 70-80% or more of the original carbohydrates from the biomass into fermentable sugars. FIGS. 7A and 7B show how enzymatic hydrolysis weight loss can depend on cellulase dosage and the TTA of the green liquor pretreatment solution. FIG. 8 shows how lignin can affect enzymatic hydrolysis. FIGS. 9A, 9B, 10A, 10B, 11A, and 11B show how glucan and xylan hydrolysis can relate to cellulase dosage and TTA of the green liquor pretreatment. FIGS. 12A, 12B, 13A, 13B, 14A, and 14B show how the enzymatic hydrolysis time can affect glucan and xylan hydrolysis efficiency in green liquor pretreated pulp.

In some embodiments, the pretreated biomass is treated with enzymes for a period ranging between about 6 hours and about 96 hours (e.g., about 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, or 96 hours). In some embodiments, the enzymatic hydrolysis time is at least about 40 hours. Enzymatic hydrolysis can be performed at any suitable temperature (e.g., depending on the enzymes used). In some embodiments, the enzymatic hydrolysis is performed at between about room temperature (i.e., about 20° C.) and about 50° C. In some embodiments, the enzymatic hydrolysis can be performed at about 38° C. In some embodiments, the enzymatic hydrolysis can be performed at about 50° C.

The enzymatic hydrolysis can be carried out at between about 5 and about 10% fiber consistency (% K) or at a higher fiber consistency (e.g., between about 10% K and about 30% K) as described in the co-pending PCT International patent application titled “HIGH CONSISTENCY ENZYMATIC HYDROLYSIS FOR THE PRODUCTION OF ETHANOL (which claims priority to U.S. Provisional Patent Application Ser. No. 61/116,909). For example, in some embodiments, lignocellulose-degrading enzymes can be mixed with green liquor-pretreated biomass at a fiber consistency of about 5% K for a few minutes (e.g., between about 1-20 minutes), thickened (e.g., using a filter press) to a higher fiber consistency (e.g., between about 10% K and about 30% K) and allowed to hydrolyze for an additional period of time at the higher fiber consistency (e.g., between 1 and 3 days). Additional lignocellose-degrading enzymes can be added to the thickened mixture (e.g., about 2-3 hours following thickening). In some embodiments, an enzyme composition comprising cellulase is mixed with the fibers at the lower fiber consistency and a second enzyme composition (e.g., comprising xylanase and β-glucosidase or comprising cellulase, xylanase, and β-glucosidase) is added at the higher fiber consistency.

According to the presently disclosed methods, the combined glucan and xylan yield can be between about 50% and about 90% (e.g., about 50%, 55%, 60%, 65%, 70%, 75%, 80%, 85%, or 90%) based upon the total amount of material present in the original lignocellulosic biomass. The sugars provided by the enzymatic hydrolysis can be used as sugar, for example in the food industry or can be further transformed into other molecules.

Referring again to FIGS. 1 and 2, in some embodiments, the sugars (e.g., monomeric sugars such as glucose, mannose, xylose, etc) provided by the enzymatic hydrolysis can be fermented into an alcohol mixture using a suitable microorganism (e.g., yeast) during a fermentation step. The alcohol mixture can then be purified via distillation. Further residual water can be removed using a suitable hygroscopic material (e.g., molecular sieves). In some embodiments, the alcohol is ethanol. If the alcohol is ethanol, the ethanol can be denatured if desired, through the addition of a suitable additive (e.g., methanol, isopropyl alcohol, acetone, methyl ethyl ketone, or methyl isobutyl ketone). The alcohol can be used directly as a fuel or mixed with another component to provide a fuel mixture. For example, ethanol produced using a green liquor pretreatment step can be mixed in a biofuel composition with gasoline to provide a gasohol.

### EXAMPLES

The following Examples have been included to provide illustrations of the presently disclosed subject matter. In light of the present disclosure and the general level of skill in the art, those of skill will appreciate that the following Examples are intended to be exemplary only and that numerous changes, modifications and alterations can be employed without departing from the spirit and scope of the presently disclosed subject matter.

### Example 1

### Green Liquor Pretreatment of Lignocellulosic Biomass

Hardwood chips were mixed with green liquor (i.e., aqueous alkaline solution comprising sodium sulfate and sodium carbonate) in a pressure vessel such that the ratio of liquor to chips was approximately 4. The vessel was heated to 160° C. The TTA of the green liquor was varied from 4-20%. Following green liquor pretreatment for a period of time, the chips were removed from solution and weighed to determine the amount of material that had dissolved during the pretreatment. The pH of the black liquor (i.e., the used green liquor) was also measured. Chip yield and black liquor pH varied according to TTA of the green liquor that was used. See FIG. 3.

The green liquor pretreated chips were analyzed for polysaccharide and lignin content to determine the effects of green liquor pretreatment on the chemical content of the chips. The results of this analysis for green liquor pretreated pulps pretreated with green liquor at 4% (GL-04), 8% (GL-08), 12% (GL-12), 16% (GL-16), and 20% (GL-20) TTA are shown in Table 1, below, as well as in FIGS. 4, 5A and 5B. FIG. 6 shows the amount of lignin, xylan and mannan found dissolved in the spent green liquor. The percentage of glucan in the pretreated chips did not vary greatly in response to changes in green liquor TTA. Further, comparing the sugar amounts in the pretreated chips with untreated chips indicated that little to no alkaline degradation of cellulose occurred. Wood and pulp polysaccharide and lignin content can be measured by any suitable method, as would be readily understood by one of ordinary skill in the art upon review of the instant disclosure. Such measurements can be performed, for instance, according to analytical procedures available from the National Renewable Energy Laboratory (NREL, Golden, Colo., United States of America) and/or the Technical Association of the Pulp and Paper Industry (TAPPI, Norcross, Ga., United States of America), among others. For example, polysaccharide content in a wood or pulp sample can be measured by sulfuric acid hydrolysis of given amount of a pulp or wood sample, followed by analysis of the resulting sugars, to calculate the amount of corresponding polysaccharide originally present in the wood or pulp.

TABLE 1
Polysaccharides in Wood and Green Liquor Pretreated Pulp.

| | Polysaccharide (%) | | | | Lignin (%) | | | |
|---|---|---|---|---|---|---|---|---|
| | | | man- | | Kla- | Acid | | |
| Sample | glucan | xylan | nan | Sum^{a} | son | Soluble | Sum^{b} | Total^{c} |
| wood | 47.8 | 16.3 | 2.2 | 66.3 | 22.7 | 4.0 | 26.7 | 93.0 |
| GL-04 | 48.6 | 14.5 | 1.8 | 64.8 | 20.5 | 3.5 | 24.0 | 88.8 |
| GL-08 | 46.2 | 12.3 | 0.9 | 59.5 | 18.5 | 3.0 | 21.4 | 80.9 |
| GL-12 | 46.9 | 12.5 | 0.5 | 59.0 | 16.4 | 2.6 | 19.0 | 79.0 |
| GL-16 | 47.8 | 11.7 | 0.4 | 60.0 | 15.7 | 2.3 | 18.0 | 78.0 |
| CL-20 | 46.8 | 11.9 | 0.4 | 59.2 | 14.8 | 2.3 | 17.1 | 76.3 |

^{a}sum of glucan, xylan and mannan percentages
^{b}sum of klason and acid soluble lignan percentages
^{c}sum of glucan, xylan, mannan, klason lignin, and acid soluble lignin percentages

### Example 2

### Enzymatic Hydrolysis of Green Liquor Pretreated Liqnocellulosic Materials

Hardwood chips that had been pretreated with green liquor having TTAs of between 4 and 20 as described above in Example 1 were subjected to enzymatic hydrolysis using cellulase as described hereinabove. The amount of cellulase used varied from 5-40 FPU/gram of pretreated biomass.

Following enzymatic hydrolysis, remaining solids were collected from each sample and weighed. The weights of the remaining solids were compared to the weights of the pretreated pulps or the original wood chips as an indicator of enzymatic hydrolysis efficiency. Results for enzymatic hydrolysis of green liquor pretreated pulps pretreated with green liquor at 4% (GL-04), 8% (GL-08), 12% (GL-12), 16% (GL-16), and 20% (GL-20) TTA and calculated based on the weight of the pretreated pulp are shown in Table 2, below, and in FIG. 7A. Results based on weight of the original wood are shown in Table 3, below, and in FIG. 7B. FIG. 8 shows how the amount of lignin remaining in the pretreated biomass and enzyme dosage affects weight loss due to enzymatic hydrolysis.

TABLE 2
Enzymatic Hydrolysis Weight Loss (%) Based on Pulp Weight.

| Enzyme | | | | | |
|---|---|---|---|---|---|
| Dose | Sample Pretreatment | | | | |
| (FPU/gm) | GL-04 | GL-08 | GL-12 | GL-16 | GL-20 |
| 5 | 21.5 | 31.4 | 37.9 | 39.8 | 44.1 |
| 10 | 26.4 | 37.9 | 47.7 | 54.2 | 59.3 |
| 20 | 31.1 | 44.8 | 57.6 | 65.4 | 68.3 |
| 40 | 36.5 | 49.0 | 60.9 | 66.8 | 69.6 |

TABLE 3
Enzymatic Hydrolysis Weight Loss (%) Based on Wood Weight.

| Enzyme | | | | | |
|---|---|---|---|---|---|
| Dose | Sample Pretreatment | | | | |
| (FPU/gm) | GL-04 | GL-08 | GL-12 | GL-16 | GL-20 |
| 5 | 19.8 | 25.8 | 30.2 | 31.2 | 33.9 |
| 10 | 23.5 | 31.1 | 38.0 | 42.4 | 45.7 |
| 20 | 27.7 | 36.9 | 45.9 | 51.3 | 52.6 |
| 40 | 32.5 | 40.3 | 48.6 | 52.3 | 53.6 |

Enzymatic hydrolysis results can also analyzed by calculating the amount of hydrolysis observed for individual polysaccharides. Thus, the amount of glucan and xylan hydrolysis effected by the cellulase in the green liquor pretreated pulps was also determined. The amounts of polysaccharides hydrolyzed can be measured by any suitable method, as would be readily understood by one of ordinary skill in the art upon review of the instant disclosure, including, for example, methods available from the National Renewable Energy Laboratory (NREL, Golden, Colo., United States of America) and/or the Technical Association of the Pulp and Paper Industry (TAPPI, Norcross, Ga., United States of America).

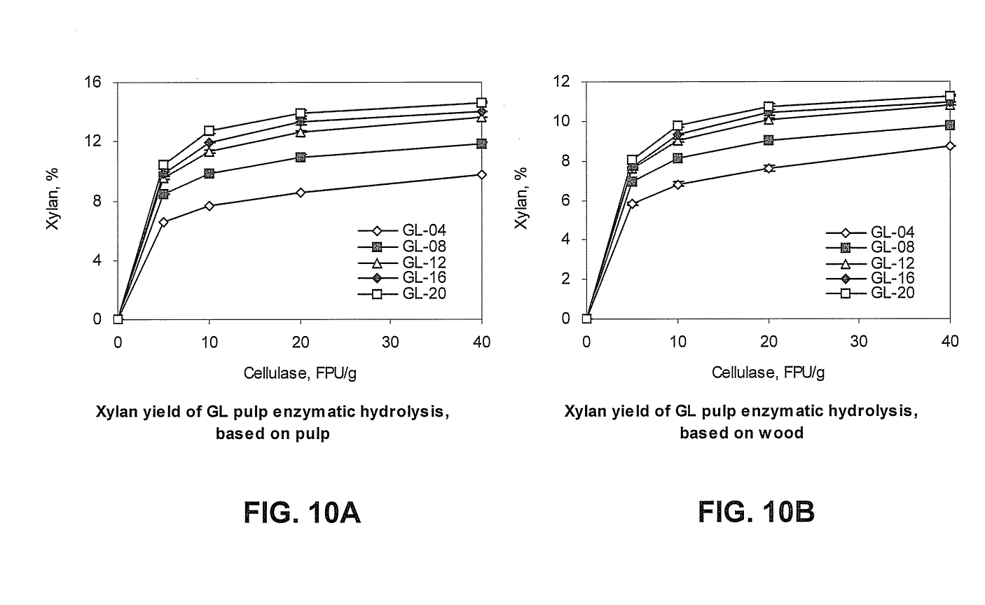

FIGS. 9A and 9B show how TTA charge and enzyme dosage affects glucan yield based on pretreated pulp content and original wood content, respectively. Data for FIGS. 9A and 9B is also provided in Tables 4 and 5, below. FIGS. 10A and 10B show how TTA charge and enzyme dosage affects xylan yield based on pretreated pulp content and original wood content, respectively. Data for FIGS. 10A and 10B is also provided in Tables 6 and 7, below. 11A and 11B show how TTA charge and enzyme dosage affects combined glucan and xylan yield.

TABLE 4
Glucan Hydrolysis (%) Based on Pulp Glucan Content.

| Enzyme | | | | | |
|---|---|---|---|---|---|
| Dose | Sample Pretreatment | | | | |
| (FPU/gm) | GL-04 | GL-08 | GL-12 | GL-16 | GL-20 |
| 5 | 14.8 | 22.5 | 28.1 | 30.3 | 32.1 |
| 10 | 19.4 | 29.2 | 37.9 | 41.9 | 44.7 |
| 20 | 24.1 | 26.1 | 47.0 | 51.3 | 51.8 |
| 40 | 29.9 | 41.0 | 51.6 | 53.1 | 54.0 |

TABLE 5
Glucan Hydrolysis (%) Based on Wood Glucan Content.

| Enzyme | | | | | |
|---|---|---|---|---|---|
| Dose | Sample Pretreatment | | | | |
| (FPU/gm) | GL-04 | GL-08 | GL-12 | GL-16 | GL-20 |
| 5 | 13.2 | 18.5 | 22.4 | 23.7 | 24.7 |
| 10 | 17.3 | 24.0 | 30.2 | 32.7 | 34.4 |
| 20 | 21.6 | 29.6 | 37.5 | 40.2 | 39.9 |
| 40 | 26.7 | 33.7 | 41.1 | 41.6 | 41.6 |

TABLE 6
Xylan Hydrolysis (%) Based on Pulp Xylan Content.

| Enzyme | | | | | |
|---|---|---|---|---|---|
| Dose | Sample Pretreatment | | | | |
| (FPU/gm) | GL-04 | GL-08 | GL-12 | GL-16 | GL-20 |
| 5 | 6.5 | 8.5 | 9.5 | 9.8 | 10.4 |
| 10 | 7.6 | 9.8 | 11.3 | 11.9 | 12.7 |
| 20 | 8.5 | 11.0 | 12.7 | 13.3 | 13.9 |
| 40 | 9.8 | 11.8 | 13.6 | 14.0 | 14.6 |

TABLE 7
Xylan Hydrolysis (%) Based on Wood Xylan Content.

| Enzyme | | | | | |
|---|---|---|---|---|---|
| Dose | Sample Pretreatment | | | | |
| (FPU/gm) | GL-04 | GL-08 | GL-12 | GL-16 | GL-20 |
| 5 | 5.8 | 7.0 | 7.6 | 7.7 | 8.0 |
| 10 | 6.8 | 8.1 | 9.0 | 9.3 | 9.8 |
| 20 | 7.6 | 9.0 | 10.1 | 10.4 | 10.7 |
| 40 | 8.7 | 9.7 | 10.8 | 11.0 | 11.3 |

The amount of time green liquor pretreated pulps (e.g., 12% TTA GL (GL-12) and 16% TTA GL (GL-16)) were contacted with cellulase was also varied from 6 hours to 96 hours to determine the effects of enzymatic hydrolysis time on hydrolysis efficiency. The results are shown in FIGS. 12A, 12B, 13A, 13B, 14A, and 14B.

Green liquor pretreatment enhances enzymatic hydrolysis in lignocellulosic biomass. More than 75% of polysaccharides in the wood samples tested can be recovered as monomers when the wood is pretreated with green liquor at TTA charges of 16-20% followed by enzymatic hydrolysis at enzyme dosages between 20 and 40 FPUs. In comparison, pretreatments involving heating to 160° C. only or treatment with sodium carbonate did not appear to be effective in enhancing subsequent enzymatic hydrolysis of lignocellulosic materials to provide fermentable sugars. Pretreatment with acetic acid followed by green liquor pretreatment led to higher enzymatic hydrolysis efficiencies than pretreatment with green liquor only, however the total sugar yield of acetic acid-green liquor pretreated pulps was slightly lower. Without being bound to any one theory, it is believed that pretreatments using acetic acid lead to reduction in xylan yield.

### Example 3

### Oxygen Delignification and/or Refining Prior to Enzymatic Hydrolysis

Prior to enzymatic hydrolysis, green liquor treated pulp can be subjected to either oxygen delignification, refining, or both.

Oxygen Delignification: Oxygen delignification of green liquor treated pulp was carried out in a 2.8 L reactor in an oven heated by blowing hot air. GL pulp (100 g oven dried (OD)) was treated with 2-5% NaOH on pulp at 10% consistency under 100 psig oxygen pressure and at 110° C. for 60 min (excluding time to temperature of 45 min). After delignification, the pulp was washed with cold water, centrifuged and fluffed.

Refining: Pulp (30 g OD) was refined in a PFI mill at 10% consistency for 9,000 revolutions. After refining, pulp was collected and hydrolyzed with enzyme without washing. Alternatively, refining can be performed using a paper machine refiner.

Table 8 below shows the effect of oxygen delignification and/or refining on enzymatic hydrolysis of green liquor pretreated pulp (e.g., 20% TTA GL (GL20), 16% TTA GL (GL16), and 12% TTA GL (GL12)). For softwood pulp pretreated with green liquor with a TTA of 16%, oxygen delignification or refining prior to enzymatic hydrolysis increase the enzymatic conversion rate from about 44% to about 52% or 59%, respectively. When both refining and oxygen delignification were employed following green liquor pretreatment, over 76% of the total polysaccharides in the wood were converted to fermentable sugars. This increase in enzymatic conversion rate is particularly noteworthy when compared to the data for oxygen delignification or refining alone. In particular, there appears to be a synergetic effect on total sugar conversion produced by subjecting green liquor pretreated pulp to both oxygen delignification and refining.

TABLE 8
Effect of Oxygen Delignification and Refining on Enzymatic
Hydrolysis of Green Liquor Pretreated Softwood Pulp.

| | | | Total sugar yield in | | |
|---|---|---|---|---|---|
| | Weight loss (%) | | hydrolysate (%) | | Sugar |
| | On | On | On | On | conversion* |
| | pulp | Wood | pulp | wood | (%) |
| GL20 | 38.4 | 29.4 | 37.7 | 28.8 | 46.5 |
| GL16 | 33.4 | 25.7 | 35.6 | 27.4 | 44.3 |
| GL12 | 30.8 | 24.2 | 34.0 | 26.7 | 43.6 |
| GL20-R | 51.0 | 39.0 | 51.3 | 38.9 | 63.5 |
| GL16-R | 47.2 | 36.3 | 47.4 | 37.0 | 59.0 |
| GL12-R | 43.8 | 34.4 | 42.6 | 33.1 | 51.1 |
| GL20-O | 49.3 | 35.6 | 47.0 | 34.0 | 54.9 |
| GL16-O | 44.5 | 32.8 | 43.3 | 32.0 | 51.7 |
| GL12-O | 35.1 | 27.0 | 33.2 | 28.6 | 41.4 |
| GL20-O-R | 65.9 | 47.7 | 64.2 | 46.4 | 75.3 |
| GL16-O-R | 66.4 | 49.0 | 63.6 | 46.9 | 76.3 |
| GL12-O-R | 59.4 | 45.7 | 60.0 | 46.2 | 74.6 |

*Sugar conversion = Weight loss (on wood)/Total sugar in wood × 100%
R = refining
O = Oxygen delignification

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method of producing an alcohol from a lignocellulosic biomass, the method comprising:
providing lignocellulosic biomass;
contacting the lignocellulosic biomass with an alkaline composition comprising sodium sulfide and sodium carbonate to provide a pretreated lignocellulosic mixture;
contacting the pretreated lignocellulosic mixture with an enzyme composition to provide a fermentable sugar mixture; and
fermenting the fermentable sugar mixture to provide an alcohol.

2. The method of claim 1, wherein the lignocellulosic biomass is selected from the group consisting of herbaceous material, agricultural residues, forestry residues, municipal solid wastes, waste paper, pulp and paper mill residues, or a combination thereof.

3. The method of claim 2, wherein the lignocellulosic biomass is selected from the group consisting of corn stover, straw, bagasse, miscanthus, sorghum residue, switch grass, bamboo, water hyacinth, hardwood, hardwood chips, softwood chips, hardwood pulp, and softwood pulp.

4. The method of claim 1, wherein contacting the lignocellulosic biomass with an alkaline composition comprises providing a charge comprising total titratable alkali (TTA) ranging from about 4% to about 25%.

5. The method of claim 4, wherein contacting the lignocellulosic biomass with an alkaline composition comprises providing a charge comprising TTA ranging from about 12% to about 20%.

6. The method of claim 5, wherein the TTA is about 16%.

7. The method of claim 1, wherein the alkaline composition has a sulfidity ranging from about 5% to about 50%.

8. The method of claim 7, wherein the sulfidity is about 25%.

9. The method of claim 1, wherein the alkaline composition has a pH of between about 8 and about 9.5.

10. The method of claim 1, wherein contacting the lignocellulosic biomass with the alkaline composition takes place at a temperature of between about 100° C. and about 220° C.

11. The method of claim 10, wherein the temperature is between about 160° C. and about 170° C.

12. The method of claim 1, wherein contacting the lignocellulosic biomass with the alkaline composition takes place for a period of time between about 0.25 hours and about 4 hours.

13. The method of claim 1, wherein contacting the lignocellulosic biomass with the alkaline composition takes place in a carbon steel pressure vessel.

14. The method of claim 1, further comprising removing one or more of non-cellulosic material, non-fibrous cellulosic material, and non-degradable cellulosic material from the pretreated lignocellulosic mixture prior to contacting the pretreated lignocellulosic mixture with the enzyme composition.

15. The method of claim 14, wherein the removing comprises washing the pretreated lignocellulosic mixture to remove dissolved substances.

16. The method of claim 15, wherein the dissolved substances are selected from the group consisting of lignin, degraded cellulose compounds, alkaline chemical compounds, and combinations thereof.

17. The method of claim 16, wherein the lignin is collected and used as an energy source.

18. The method of claim 17, wherein the lignin is used as an energy source to provide heat during a distillation or other step during the production of alcohol.

19. The method of claim 16, wherein the alkaline chemical compounds are recycled.

20. The method of claim 14, wherein the removing comprises subjecting the pretreated lignocellulosic mixture to oxygen delignification prior to contacting the pretreated lignocellulosic mixture with the enzyme composition.

21. The method of claim 1, further comprising refining solid material in the pretreated lignocellulosic mixture prior to contacting the pretreated lignocellulosic mixture with the enzyme composition, thereby reducing the solid material in size.

22. The method of claim 21, wherein the refining provides bundles of cellulose fibers, single cellulose fibers, fragments of single cellulose fibers, or a combination thereof.

23. The method of claim 21, wherein the refining is performed using a mechanical refiner.

24. The method of claim 1, further comprising subjecting the pretreated lignocellulosic mixture to oxygen delignification and refining solid material in the pretreated lignocellulosic mixture prior to contacting the pretreated lignocellulosic mixture with the enzyme composition.

25. The method of claim 1, wherein the lignocellulosic biomass comprises hardwood chips or softwood chips, and contacting the lignocellulosic biomass with the alkaline composition provides a pretreated lignocellosic mixture comprising about 80% of cellulosic material from the lignocellulosic biomass present as undissolved cellulosic fibers or fragments thereof.

26. The method of claim 1, wherein the pretreated lignocellulosic mixture comprises between about 17% and about 24% residual lignin.

27. The method of claim 1, wherein the enzyme composition comprises between about 5 filter-paper units (FPU) and about 40 FPU cellulase per gram of pretreated lignocellulosic material.

28. The method of claim 27, wherein the enzyme composition comprises between about 20 FPU and about 40 FPU cellulase per gram of pretreated lignocellulosic material.

29. The method of claim 1, wherein cellulosic material from the pretreated lignocellulosic mixture is contacted with the enzyme composition for a period of time ranging between about 6 hours and about 96 hours.

30. The method of claim 29, wherein the period of time ranges between about 40 hours and about 96 hours.

31. The method of claim 1 wherein contacting the pretreated lignocellulosic mixture provides a fermentable sugar mixture comprising a combined glucan and xylan yield of between about 50% and about 90% based on total cellulosic material originally present in the lignocellulosic biomass.

32. The method of claim 1, wherein fermenting comprises contacting the fermentable sugar mixture with a microorganism to provide an alcohol mixture and distilling the alcohol mixture to provide the alcohol.

33. The method of claim 32, wherein the microorganism is yeast.

34. The method of claim 32, further comprising dehydrating the alcohol to remove residual water.

35. The method of claim 1, wherein the alcohol is ethanol.

36. A composition comprising an alcohol prepared from lignocellulosic biomass using a method that comprises pretreatment of the lignocellulosic biomass with an alkaline solution comprising sodium sulfide and sodium carbonate.

37. The composition of claim 36, wherein the alcohol is ethanol.

38. The composition of claim 37, wherein the composition comprises 95% or greater ethanol.

39. The composition of claim 37, wherein the composition comprises a fuel mixture comprising ethanol and gasoline.
